(19) United States
(12) Patent Application Publication
(10) Pub. No.: US 2007/0027367 A1
(43) Pub. Date: Feb. 1, 2007

(54) Mobile, personal, and non-intrusive health monitoring and analysis system

(75) Inventors: Nuria Maria Oliver, Seattle, WA (US); Fernando Flores-Mangas, Mexico D.F. (MX); Dane Michael Howard, Sammamish, WA (US); Eric G. Lang, Yarrow Point, WA (US); Russell I. Sanchez, Medina, WA (US); Michael Jack Sinclair, Kirkland, WA (US); Alfred Yong-Hock Tan, Bellevue, WA (US); Ralph Donald Thompson III, Sammamish, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Microsoft Corporation, Redmond, WA (US)

(21) Appl. No.: 11/195,338

(22) Filed: Aug. 1, 2005

(51) Int. Cl. A61B 5/00; A61B 5/02; A61B 5/04; A61B 10/00

(52) U.S. Cl. 600/300; 128/903; 600/500; 600/549; 600/544; 600/565; 600/485

(57) ABSTRACT

An open architecture, wireless personal area network for receiving, storing, processing, displaying and communicating physiological data. The wireless personal area network may include a personal server, such as a cellular phone, and a plurality of sensors to monitor physiological signs, the user's motion, the user's orientation, and environmental factors. The sensors wirelessly provide data to the personal server, which may store, process, display, and communicate the data. An open architecture allows additional sensors to join the network without rendering the personal server irrelevant.

### TECHNICAL FIELD

Open architecture, wireless personal area network for receiving physiological data.

### BACKGROUND

Currently, recording an individual's physiological signs that does not include full time care at a hospital, involves equipment that is both intrusive and usually only provides spot information. Generally, if an individual wishes to have physiological signs monitored, the individual must visit a physician or health care provider facility. Because the individual is taken out of his or her normal environment, the individual may be under stress, and the physiological information that is collected may not be representative of the individual for the great majority of the time that the individual is away from the physician. Furthermore, any physiological information that is gathered at a remote facility is generally only collected for a short, limited amount of time. Any physiological sign monitoring system that is currently in existence requires physiological sensors that are uniquely configured to operate only within a closed, specific environment, not within an open networked environment. The intrusive nature of physiological sensors prevents individuals from gaining knowledge of their health. Lack of quantitative knowledge about the condition of one's body limits intelligent and informed decision-making about lifestyle choices and inhibits disease prevention and one's general health.

### SUMMARY

This Summary is provided to introduce a selection of concepts in a simplified form that are further described below in the Detailed Description. This Summary is not intended to identify key features or essential features of the claimed subject matter, nor is the Summary to be used as an aid in determining the scope of the claimed subject matter.

Emerging technologies have made it possible to create the personal area network (PAN) and the wireless personal area network (WPAN). A personal area network, wireless or not, is a computer network composed of various devices within close proximity to one person, wherein the devices are able to communicate with one another. The personal area network may include a master device able to communicate with a plurality of slave devices, which must first be authenticated, in order to enable further communication between the master device and the slave device. In the Detailed Description, a wireless personal area network having an open architecture is described. An open architecture is a system design strategy incorporating published specifications so that third parties may develop software and hardware to be added on to the system or device. The wireless personal area network includes a plurality of sensors that may monitor physiological signs in real time. Other sensors that may be part of the wireless personal area network include sensors that may not monitor physiological signs. Non-physiological sensors may monitor a person's motion, the environment, or the person's orientation. The “master” device in the wireless personal area network may be a mobile, personal computing device, such as a cell phone, personal digital assistant (PDA), laptop computer, or other computing device. All mobile, personal devices may be referred to simply as computing devices or computer. The computing device and the sensors in the wireless personal area network are equipped with devices having a common communications protocol to provide an open architecture. Thus, any sensor that includes the common communications protocol may join the wireless personal area network. The wireless personal area network allows data collection from multiple sensors. Wireless encryption protocol to protect wirelessly transmitted data may also be provided. A set of wireless sensors are attached, worn, or even embedded at different locations on the body. Since sensors share a common radio protocol, individual sensors can be added, replaced, or removed to suit the needs of the user. This feature enables the wireless personal area network to grow, without rendering the master device irrelevant, since other sensors may subsequently join in the wireless personal area network. Accordingly, one master device may communicate with a plurality of sensors that are within the network, provided that the sensor is equipped with a communications protocol similar to the master device.

The wireless personal area network described below may provide an individual with the ability to observe real-time measurements of their body condition and their environment, and through storage and intelligent analysis of the data, the individual is provided with trend analysis and recommended behavioral changes. The information is instrumental in assisting the individual to achieve personal health goals such as weight loss, increased energy and stamina, increased life span, increased physical capability, as well as management and monitoring of chronic disease and the prevention of disease and other bodily damage.

### DESCRIPTION OF THE DRAWINGS

The foregoing aspects and many of the attendant advantages will become more readily appreciated as the same become better understood by reference to the following detailed description, when taken in conjunction with the accompanying drawings, wherein:

FIG. 1 is a schematic illustration of a wireless, personal area network for receiving physiological data;

FIG. 2 is a flow diagram of a method for receiving data in a wireless personal area network;

FIG. 3 is a diagrammatical illustration of a wireless, personal area network for receiving physiological data;

FIG. 4 is a schematic illustration of modules for a computing device in a wireless, personal area network;

FIG. 5 is a diagrammatical illustration of a portion of a wireless, personal area network for receiving physiological data; and

FIG. 6 is a flow diagram of an algorithm for determining sleep apnea.

### DETAILED DESCRIPTION

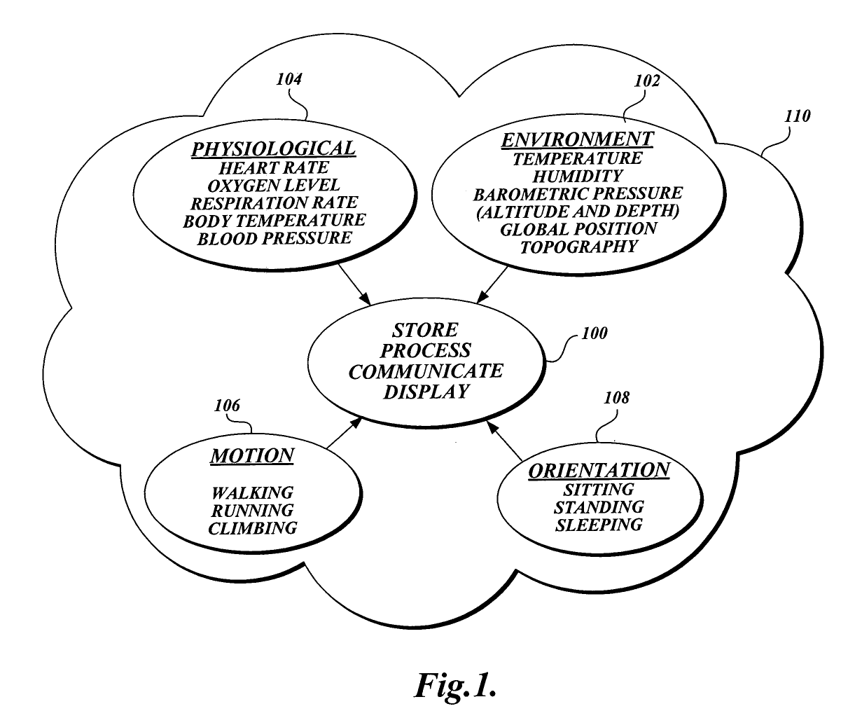

FIG. 1 shows a schematic illustration of an open architecture, wireless, personal area network 110 for receiving, at least, physiological data. At the center of the network 110 is computing device 100, which is capable of any one process of receiving, storing, processing, communicating, and displaying a multitude of data and information gathered from sensors in proximity to a person. Sensors in proximity to a person may be located on a person, close to a person, or on a device wearable by the person. The sensors may be categorized broadly as environmental sensors 102, physiological sensors 104, motion sensors 106, and orientation sensors 108. At least one physiological sensor forms a part of the system and network. Environmental sensors 102 may measure any one or more of environmental factors, including, but not limited to, temperature, humidity, barometric pressure, global position, and topography. Physiological sensors 104 may measure any one or more of physiological parameters, including, but not limited to heart rate, blood oxygen level, respiration rate, body temperature, cholesterol level, blood glucose level, galvanic skin response, EEG, and blood pressure. Motion sensors 106 can be used for determining the person's activity, including whether the person is walking, running, or climbing. Orientation sensors 108 determine the position of the person, including whether the person is sitting, standing, or sleeping. It is to be appreciated that the naming of sensors for specific purposes is merely to illustrate representative embodiments of the invention, and should not be construed to limit the invention to anyone specific embodiment. Combining the information gathered from various sensors over a wireless, personal area network may lead to intelligent choices concerning all issues of a person's health.

Specific features of the open architecture, wireless personal area network may include operation within a low bandwidth, and being non symmetric, meaning that data sensors may transmit to the master device based on commands from the master device to the sensors. The open architecture, wireless personal area network may incorporate high precision, high accuracy, high reliability, and low power sensors, and have noise compensation for motion, temperature, moisture, and audio. The open architecture, wireless personal area network may include high security and privacy features, and deliver data on demand. Sensors may be stable at temperatures near to the body. The open architecture, wireless personal area network may include dynamic sensor selection depending on context or application. Sensors may include a thermal switch that can be activated by body temperature through body contact. Sensors may synchronize transmission of data or other activity based on a physiological sign, such as heart rate. Sensors may transmit data continuously, or data may be held in a buffer in cache memory or data may periodically be sent in bursts.

Computing device 100 and the sensors in the wireless personal area network 110 operate in an open environment and, as such, the computing device 100, as the master device, will be able to recognize and communicate with each sensor brought into the network 110 through the use of an common communications protocol, such as, but not limited to a BLUETOOTH, ZIGBEE, and 802.11 communications protocol. A wireless, personal area network for monitoring, at least, physiological signs provides the ability to measure continuously, or at least for extended periods of time, physiological signs that will be representative of the person in his or her normal environment. Furthermore, as the sensors are communicating in a personal area network, power requirements for sensors will be kept low.

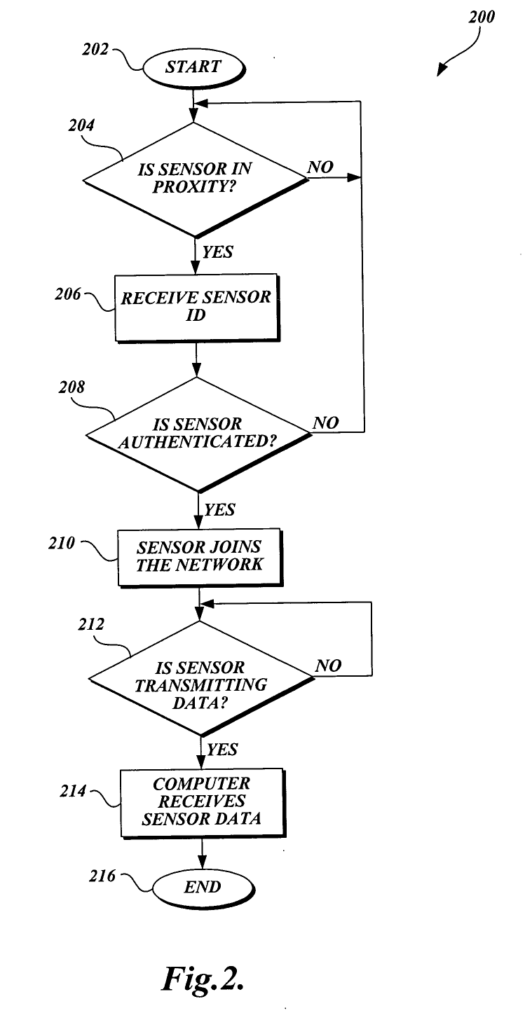

Referring to FIG. 2, a flow diagram of an embodiment of a method 200 for receiving data in an open architecture, wireless, personal area network is illustrated. Acquisition of data in a wireless personal area network having physiological sensors may be used to record, store and analyze the data to detect unusual events, identify patterns of behavior, and help users achieve specific targets of physical activity. In one embodiment, users of the system may select any one of a number of different type of sensors, including sensors that may measure physiological signs, the type of motion, the person's orientation, and the person's environmental factors. Each sensor is provided with the ability to communicate in the personal area network. The selected sensors may communicate with the computing device 100, such as a cellular phone, PDA, or laptop, which may store and analyze the data in a number of different manners to detect patterns of behavior and unusual events that would trigger a visit to the health care provider for further diagnosis and treatment. Method 200 starts with the start block 202. In block 202, computing device 100 is awaiting to receive a signal from a sensor within proximity of it. From block 202, method 200 enters decision block 204. In block 204, a determination is made whether there is a sensor within proximity of the computing device 100. If the determination in decision block 204 is “no”, meaning that there is no sensor in proximity, the method 200 continues to wait. If the determination in decision block 204 is “yes”, meaning that the computing device 100 has detected a sensor within the broadcast range, the method 200 enters block 206. In block 206, the sensor transmits the sensor identification (ID) to, and the sensor ID is received by the computing device 100. It is possible that more than one sensor may be in proximity at one time. The communications protocol may establish an orderly series of discovery rules that may sequentially discover each sensor in the network. From block 206, the method 200 enters decision block 208. In block 208, a determination is made by the computing device 100 whether the sensor ID is authenticated, meaning whether the sensor is granted permission to join the network. A series of authentication rules specific to the communications protocol used may determine whether the sensor is permitted to join the network. If the determination in decision block 208 is “no”, meaning that the sensor is not authenticated, the method 200 returns to wait for the next sensor to be in proximity to the computing device 100, block 204. If the determination in decision block 208 is “yes”, meaning that the sensor is authenticated, then the method 200 enters block 210. In block 210, the sensor joins the network 110. From block 210, the method 200 enters decision block 212. In decision block 212, a determination is made whether the sensor is transmitting data. The communications protocol may establish an orderly series of transmission rules for the orderly transmitting of data from each sensor in the network to the computing device 100 in order to establish a procedure whereby transmitted data is not lost. According to the transmission rules, each sensor may be allotted a time window for a specified period of time in which to transmit, and/or at an established time interval. Alternatively, each sensor may transmit in a different radio frequency, and the frequency may vary with each transmission. Alternatively, each sensor may transmit according to an internal clock residing with the computing device 100. In this way, a master-slave procedure is established, wherein the master device, i.e., the computing device 100 will let the slave device, i.e., the sensor, know when it is time to transmit. If the determination in decision block 212 is “no”, meaning that the sensor is not transmitting data, then the sensor waits its turn. If the determination in decision block 212 is “yes”, meaning that the transmission rules have determined that the sensor should be transmitting, and the sensor is transmitting data, the method 200 enters block 214. In block 214, the computing device 100 may receive the sensor data, which may be stored, used in an algorithm, communicated remotely, displayed locally, and/or processed in any other manner. From block 214, the method 200 enters block 216. Block 216 is a terminus block for one iteration of method 200. Method 200 may be continuously implemented by computing device 100 for each sensor that is brought in proximity to the computing device 100. The open architecture, wireless, personal area network may include one or more sensors, and may also include one or more computing devices 100. In one implementation of an open architecture networked system, the wireless, personal area network includes at least one computing device 100, and at least one sensor that may transmit physiological data.

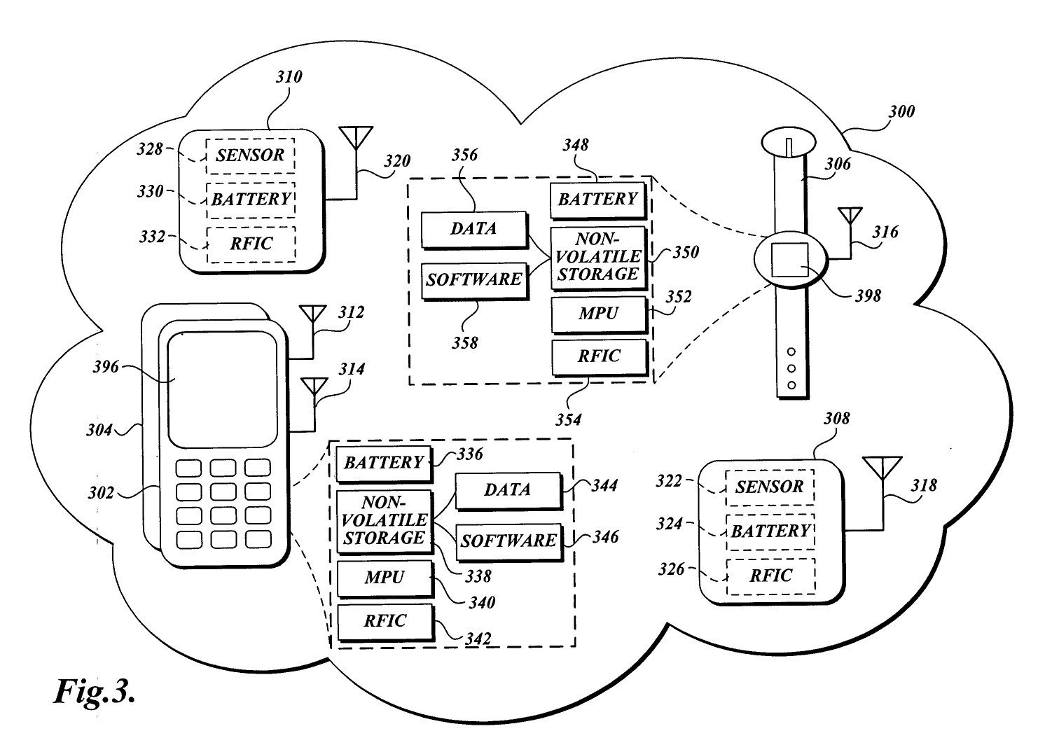

Referring now to FIG. 3, one embodiment of a wireless personal area network 300 is illustrated. In this embodiment, a mobile cellular phone 302 serves as a master device in the wireless personal area network 300. The cellular phone 302 may be connected to periphery devices 304, including, but not limited to auxiliary displays, printers, and the like. The cellular phone 302 may include, a battery 336 for power, non-volatile storage 338 for the storage of data collected from sensors 344 and for storage of software 346, a microprocessor chip (MPU) 340, a display 396 for use as a user interface (UI), a radio frequency integrated circuit (RFIC) 342 with radio frequency antenna 314 for communication in the wireless personal area network 300, and a microwave frequency antenna 312 for communication in a cellular telephone network. Master devices may also be implemented as any wearable device, such as, but not limited to a wrist device 306. Wrist device 306 may include, a battery 348 for power, non-volatile storage 350 for the storage of data collected from sensors 356 and for storage of software 358, a MPU 352, a UI 398, a RFIC 354, and a radio frequency antenna 316 for communication in the wireless personal area network 300.

FIG. 3 also illustrates a number of sensor devices, 308 and 310. Sensor device 308 includes a sensor 322 to measure the variable of interest, a battery 324 to power the sensor device, and a RFIC 326 with radio frequency antenna 318 to communicate in the wireless personal area network 300. Sensor device 310 includes a sensor 328 to measure the variable of interest, a battery 330 to power the sensor device, and a RFIC 332 with radio frequency antenna 320 to communicate in the wireless personal area network 300. Because the sensor devices 308 and 310 employ a low power radio frequency communication interface, the life of batteries 324 and 330 may be extended. The RFICs 326 and 332 provide the wireless communication interface. Representative examples, include, but are not limited, to 802.15.4 (ZIGBEE), 802.15.1 (BLUETOOTH), 802.15.3 (UWB), 802.11x (Wimax). The batteries 324 and 330 supply power to the sensor devices 308 and 310, respectively.

Both UIs 396 and 398 are for presenting information to the user, in either text, or graphics, for example, and also for responding to user commands and/or receiving user commands. The non-volatile storage media 338 and 350 retain the data 344 and 356, respectively, from the sensor devices 302 and 306, and the software 346 and 358. The MPUs 340 and 352 execute the software 346 and 358 for collecting data, storing data, performing data analysis, managing the UIs 396 and 398, and serve as the interface with the RFICs 342 and 354. The software 346 and 358 may provide functions for presenting real-time data values to the user via a display. The software 346 and 358 may compile and present aggregated health indices providing the user a quantitative measure of trends related to physical health, such as life expectancy. The software 346 and 358 may ascertain and present recommendations for efficiently progressing towards health goals specified by the user. The RFICs 342 and 354 provide the wireless communication interface. Representative examples include, but are not limited to 802.15.4 (ZIGBEE), 802.15.1 (BLUETOOTH), 802.15.3 (UWB), 802.11x (Wimax). Through the RFICs 342 and 354, master devices 302 and 306 may be able to communicate with sensor devices 308 and 310. In one embodiment, sensor device 308 may be physiological sensor and sensor device 310 may sense other than a physiological sign, such as a sensor device to monitor motion, orientation, or the environment. If sensor device 310 is a motion sensor, sensor device 310 may be an accelerometer or a magnetometer. Cellular phone 302 may also communicate with the wrist-mounted device 306. Although one implementation of the open architecture wireless personal area network has been described with reference to a cellular phone as a master device, it is to be understood that the invention is not limited to any one specific implementation of a master device.

In the open architecture design described, sensor devices may be allowed to join the wireless personal area network provided that the sensor device includes a communications protocol compatible with the master device's communications protocol. In an open architecture wireless, personal area network, the master device may either be continuously or intermittently monitoring for new sensor devices to join the personal area network. Toward this end, the master device may include a discovery module for determining when a new sensor device has joined the network. The master device will be listening for radio signals at a common frequency. Similarly, the sensor device that is new to the personal area network will broadcast in the same frequency as the master device. The sensor device new to the personal area network will be broadcasting its identification number. When the master device receives a signal that the master device recognizes, the master device will interpret the identification number. The master device is pre-programmed to recognize specific identification numbers. If the identification number is recognized by the master device, the master device will allow the sensor device new to the personal area network to establish a connection to the master device, and the sensor device may begin transmitting data that the master device can receive.

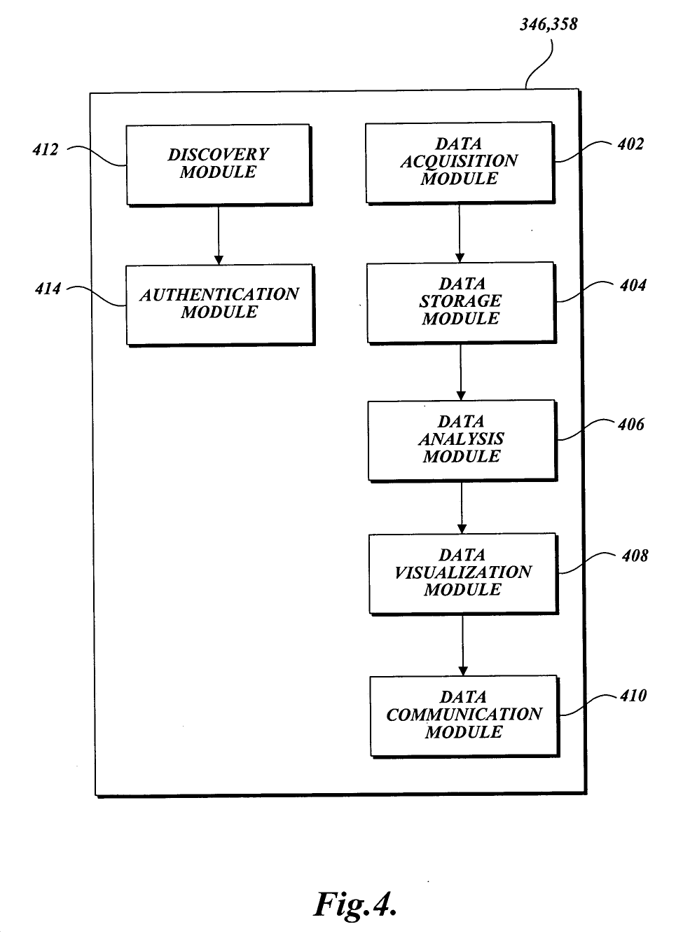

Referring now to FIG. 4, within the software components 346 and 358 of master devices 302 and 306, respectively, is a data acquisition module 402, a data storage module 404, a data analysis module 406, a data visualization module 408, a data communication module 410, a discovery module 412, and an authentication module 414. Data acquisition module 402 is provided for wirelessly interfacing with the sensor devices 308 and 310 using a standard serial port profile (SPP). The data acquisition module 402 can collect data from as many sensors as needed, and send some information to the sensors when appropriate. The data acquisition module 402 may implement transmission rules for the orderly transmission of data between master devices 302 and 306 with sensor devices 308 and 310. The data storage module 404 stores the physiological data for later processing and analysis. The data may be viewed locally; alternatively, the data may be stored for later viewing, such as at a remote location. The data analysis module 406 includes pattern recognition and machine learning algorithms for identifying patterns of behavior and anomalies in the sensor data. The data visualization module 408 is for presenting the physiological data to the user or health-care provider in an intelligible format. The data communication module 410 is for wirelessly transmitting the data to other devices, either through a radio or microwave frequency. The discovery module 412 is for implementing the discovery rules when a new sensor device is brought in proximity to the master devices 302 or 306. The authentication module 414 is for implementing the authentication rules after the sensor ID has been received by the master devices 302 or 306.

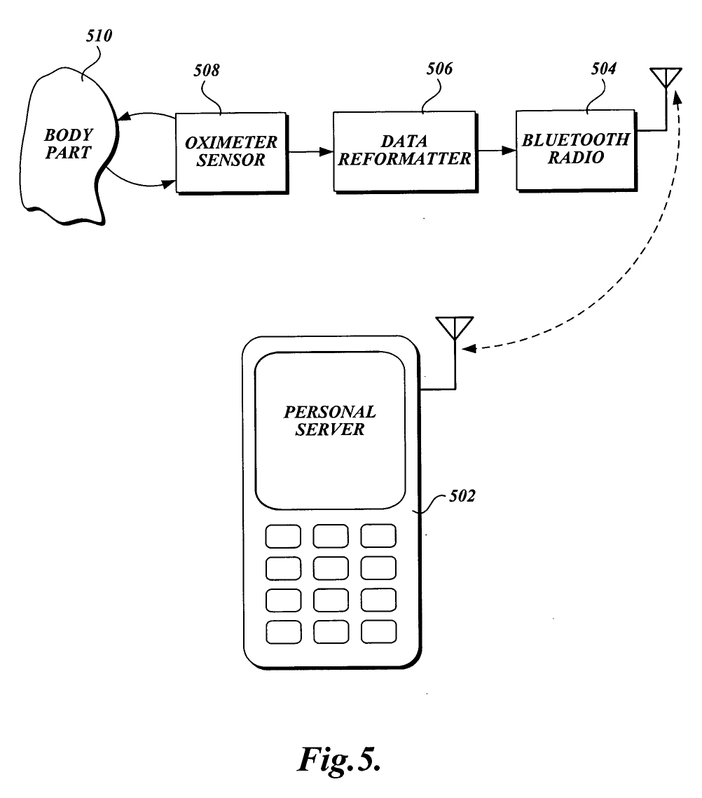

FIG. 5 is a schematic representation of one embodiment, wherein a personal server 502 communicates via a BLUETOOTH radio device 504, to an oximeter sensor 508 in contact with a body part 510. In this embodiment, a data reformatter 506 is provided to convert the signal coming from oximeter sensor 508 into a signal that can be used by the BLUETOOTH radio device 504. In this implementation, the wireless oximeter sensor 508 is a PULSEOX model no. 5500, which is a finger unit blood saturation and heart rate spot-monitor from the SPO Medical company. PULSEOX model no. 5500 is modified to be powered continuously for an indefinite period, instead of spot checking. PULSEOX model no. 5500 also is modified to extract data for recording and processing. PULSEOX model no. 5500 provides an internal 9600 baud serial digital signal containing the oximeter data plus other, probably diagnostic data. As this data may have other non-relevant characters in the bit stream, the data reformatter 506 (PIC16F873 microprocessor) is programmed to parse and reformat the data suitable for radio frequency transmission for subsequent processing, viewing and storage. The reformatted data is then sent to the small, low-powered BLUETOOTH radio chip 504 for transmission to the personal server 502. Personal server 502 has a display, which may be used to display the sensor reading in real time. In this implementation, the personal server 502 is an AUDIOVOX SMT 5600 SMART PHONE. While this implementation is described using a blood oximeter sensor, other non-skin contacting health monitoring devices could also be incorporated such as an accelerometer, gyroscope and/or magnetometer. This type of sensor may be used for detecting physical activity or angular position of the wearer which might also give the context of the activity, such as lying down, sitting up, standing, walking, or running. In one alternative implementation, these sensors may be incorporated into or mounted to the personal server 502 rather than being radio frequency linked. A wrist mounted device may also be incorporated. Besides indication of the time, the wrist mounted device may be linked to the personal server 502. This would give the user access to readily viewable data rather than recalling the data via the user interface.

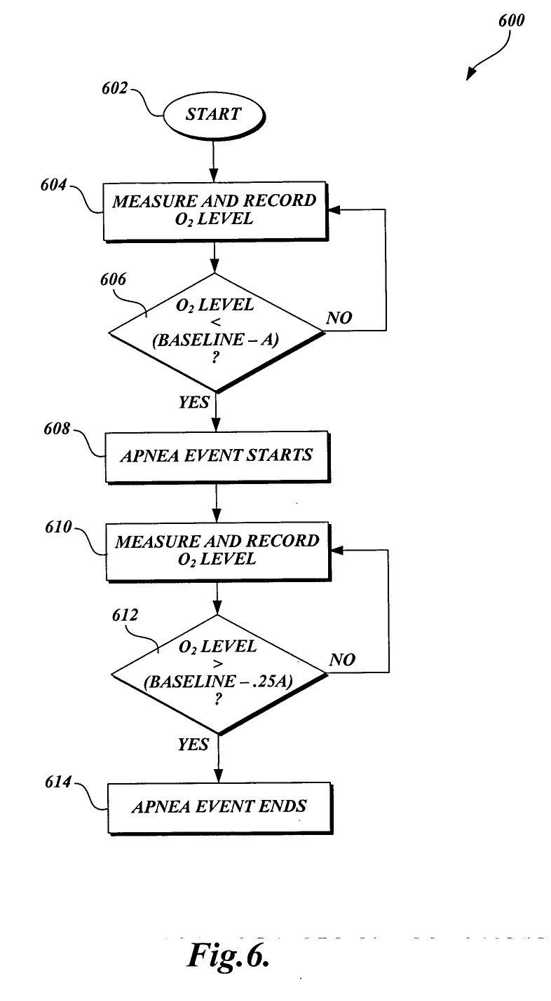

FIG. 6 is a flow diagram of a method 600 for determining whether sleep apnea is occurring using the wireless personal area network that may monitor blood oxygen. Method 600 may be used to alert, and/or to record data pertaining to the sleeping patterns of an individual for later analysis. The method 600 starts at start block 602. From start block 602, the method 600 enters block 604. Block 604 is for measuring and recording the oxygen level of an individual with a non-intrusive sensor capable of wirelessly transmitting data. After sufficient amount of oxygen level data is obtained to establish a normal baseline level, the method 600 may enter decision block 606. Decision block 606 determines whether the oxygen level is below the baseline minus a certain offset “A.” If the determination in decision block 606 is “no”, the method 600 returns to block 604, wherein the method 600 continues to measure and record the oxygen level of the individual. If the determination in decision block 606 is “yes”, the method 600 enters block 608. Block 608 is for signaling the start of an apnea event. From block 608, the method 600 enters block 610. In block 610, the method 600 continuously measures and records the oxygen level of the individual. From block 610, the method 600 enters decision block 612. In decision block 612, the method 600 determines whether the oxygen level is greater than the baseline level minus a percentage of the offset A. If the determination in decision block 612 is “no”, the method 600 returns to block 610, where the method 600 continuously measures and records the oxygen level of the individual. If the determination in decision block 612 is “yes”, the method 600 enters block 614. In block 614, the method 600 has determined that the apnea event is at an end. Although one implementation of a use for the wireless personal area network having an open architecture has been described, it is to be recognized that the invention is not limited to any one particular implementation.

While illustrative embodiments of the invention have been illustrated and described, it will be appreciated that various changes can be made therein without departing from the spirit and scope of the invention.

The embodiments of the invention in which an exclusive property or privilege is claimed are defined as follows:

1. A networked system, comprising:
a master device;
at least one sensor to monitor a physiological sign, wherein the master device and the sensor are in a wireless personal area network having an open architecture.

2. The system of claim 1, wherein the physiological sign is one of at least heart rate, oxygen level, respiration rate, body temperature, cholesterol level, blood glucose level, galvanic skin response, EEG, or blood pressure.

3. The system of claim 1, further comprising at least one sensor to monitor other than a physiological sign.

4. The system of claim 1, further comprising at least one sensor to monitor motion, orientation, or the environment.

5. The system of claim 1, wherein the master device is a cellular phone, a personal digital assistant, a computer, or a wearable device.

6. The system of claim 1, wherein the master device may store, process, communicate or display data gathered by the sensor.

7. The system of claim 1, wherein communication in the wireless personal area network is encrypted.

8. The system of claim 1, wherein the master device includes a radio frequency integrated circuit.

9. The system of claim 1, wherein the sensor includes a radio frequency integrated circuit.

10. A method of communicating physiological data over a wireless personal area network having an open architecture, comprising:
determining when a sensor device is in proximity to a master device;
receiving an identification signal from the sensor device;
authenticating the sensor device;
receiving data from the sensor device, wherein the sensor device may monitor a physiological sign.

11. The method of claim 10, wherein the physiological sign is one of at least heart rate, oxygen level, respiration rate, body temperature, cholesterol level, blood glucose level, galvanic skin response, EEG, or blood pressure.

12. The method of claim 10, further comprising receiving other than physiological data from a second sensor device.

13. The method of claim 10, further comprising receiving data to monitor motion, orientation, or the environment.

14. The method of claim 10, wherein the master device is a cellular phone, a personal digital assistant, a computer, or a wearable device.

15. The method of claim 10, further comprising determining a sleep apnea event.

16. The method of claim 10, wherein the master device includes a discovery module to determine when a sensor is in proximity to the master device.

17. The method of claim 10, wherein the master device includes an authentication module to determine when a sensor may join the wireless personal area network.

18. The method of claim 10, wherein the master device and the sensor device include a radio frequency integrated circuit.

19. A sensor device comprising a radio frequency integrated circuit with an open architecture communications protocol and a sensor to monitor a physiological sign.

20. The sensor of claim 19, wherein the sensor monitors at least one of heart rate, oxygen level, respiration rate, body temperature, cholesterol level, blood glucose level, galvanic skin response, or blood pressure.
